(19) United States
(12) Patent Application Publication
(10) Pub. No.: US 2015/0221111 A1
(43) Pub. Date: Aug. 6, 2015

(54) SYSTEM AND METHOD FOR DISPLAYING PHYSIOLOGICAL PARAMETERS WITH A SPARKLINE

(71) Applicant: COVIDIEN LP, MANSFIELD, MA (US)

(72) Inventor: John A. Battista, JR., Lafayette, CO (US)

(21) Appl. No.: 14/608,679

(22) Filed: Jan. 29, 2015

Related U.S. Application Data
(60) Provisional application No. 61/935,423, filed on Feb. 4, 2014.

(51) Int. Cl. G06T 11/20; A61B 5/08; A61B 5/0476; G06T 11/00; A61M 16/04; A61M 16/06; A61M 16/20; A61B 5/1455; A61M 16/00

(52) U.S. Cl. CPC G06T 11/206; A61B 5/14551; A61B 5/08; A61B 5/0476; A61M 16/0003; A61M 16/0051; A61M 16/04; A61M 16/06; A61M 16/20; G06T 11/001; A61M 2205/50; A61M 2205/18; A61M 2230/04; A61M 2230/40

(57) ABSTRACT

Embodiments may include displaying a sparkline representing one or more physiological parameters measured by a patient monitoring system. The sparkline may be small and incorporated on a busy graphical user interface such as a ventilator display monitor. The sparkline may be utilized to display a baseline value in conjunction with a configurable amount of values, including, but not limited to a most recent or current value and prior values in order for a caregiver to interpret any differences at a glance. The sparkline may include a background area that represents a boundary and various aspects of the sparkline may be color-coded in order to indicate whether the boundary is exceeded. A channel indicator symbol may be included in the sparkline to indicate which channel is being monitored by the patient monitoring system. Further, the sparkline may be selectable in order to bring up more detailed information.

### CROSS REFERENCE TO RELATED APPLICATION

This application claims priority from and the benefit of U.S. Provisional Application Ser. No. 61/935,423, entitled “SYSTEM AND METHOD FOR DISPLAYING PHYSIOLOGICAL PARAMETERS WITH A SPARKLINE”, filed Feb. 4, 2014, which is hereby incorporated by reference in its entirety.

### BACKGROUND

The present disclosure relates generally to medical device displays, and more particularly, to a system and method for displaying physiological data with a sparkline on medical device displays.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, medical practitioners often desire to monitor certain physiological parameters of their patients. Accordingly, a wide variety of devices have been developed for monitoring physiological parameters. Such devices provide doctors and other healthcare personnel with the information they need to provide healthcare for their patients. As a result, such monitoring devices have become an indispensable part of modern medicine. One technique for monitoring certain physiological parameters of a patient is commonly referred to as pulse oximetry, and the devices built based upon pulse oximetry techniques are commonly referred to as pulse oximeters. Additionally, ventilators may also monitor one or more breathing characteristics of a patient during ventilation. The ventilators may use the monitored one or more breathing characteristics to determine appropriate ventilation parameters for the patient.

Medical devices, including ventilators, pulse oximeters, blood pressure apparatus, and the like, often include displays that show the various physiological parameters measured by the devices. The display screens may be encapsulated in the medical device or attached via a cable as a standalone monitor. The graphical user interfaces (GUIs) that are utilized on the displays vary widely between medical devices. In general, however, the GUIs display physiological parameters with many visual elements relative to the amount of space provided on the screens. For example, a ventilator display screen includes graphs, charts, and readings such as a positive end expiratory pressure (PEEP), tidal volume, and so forth. In addition to the actual physiological parameters being measured and displayed, the ventilator GUIs typically include other virtual buttons for settings, alarms, sound, and so forth.

As a result of fitting all of these visual elements onto a screen, the ventilator GUIs become cluttered and certain values may be difficult to read in some instances. A doctor or healthcare personnel may spend more time than necessary trying to parse the graphs, charts, readings, and other information in order to find a targeted value of interest. Further, many of the elements displayed on medical devices' GUIs only display the current value of measured physiological parameters. As may be appreciated, there is a need for improved visual elements for medical device GUIs.

### BRIEF DESCRIPTION OF THE DRAWINGS

Advantages of the disclosed techniques may become apparent upon reading the following detailed description and upon reference to the drawings in which:

FIG. 1 is a block diagram illustrating an embodiment of a patient monitoring system including a sensor and a pulse oximeter with a display;

FIG. 2 is a block diagram illustrating an embodiment of a patient monitoring system including sensors and a ventilator with a display;

FIG. 3 illustrates an embodiment of a ventilator graphical user interface (GUI) including a sparkline;

FIG. 4 illustrates an embodiment of a sparkline displaying trending historical physiological parameter values and a current value that exceeds a boundary;

FIG. 5 illustrates an embodiment of a sparkline displaying trending positive historical physiological parameter values and a current value that does not exceed a boundary;

FIG. 6 illustrates an embodiment of a sparkline displaying trending negative historical physiological parameter values and a current value that does not exceed a boundary;

FIG. 7 illustrates an embodiment of a sparkline displaying trending historical physiological parameter values and a current value that includes a numerical percentage;

FIG. 8 illustrates an embodiment of a sparkline displaying trending historical physiological parameters including a channel indicator;

FIG. 9 illustrates an embodiment of a sparkline displaying trending historical physiological parameters including a waveform representing prior values; and

FIG. 10 illustrates an embodiment of a screen displaying more detailed information that is accessible by clicking a sparkline on a patient monitoring system.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described in detail below, the systems and methods provided herein are directed toward a sparkline displaying physiological parameters in a patient monitoring system. Sparklines may be data-intense, design-simple, word-sized graphics, such as a very small line or bar chart. Sparklines may present variations of data over time in a simple and highly condensed way. As such, presently disclosed embodiments include one or more features capable of utilizing a small sparkline to display the current value of physiological parameter(s) as well as the historical trend of the physiological parameter(s) over a configurable amount of time. Further, the embodiments disclosed may include features capable of configuring color coding aspects of the sparkline to indicate when configurable boundaries have or have not been exceeded. Other features of certain embodiments may include a channel indicator to represent which physiological channel (e.g., region of the body) of a patient is being monitored. Further, some embodiments may include features of a sparkline that allow a caregiver to click on the sparkline in order to drill-down and see more details of the physiological parameters. The present disclosure focuses on utilizing the sparkline in a ventilator graphical user interface (GUI); however, incorporating the sparkline into other patient monitoring systems is envisioned.

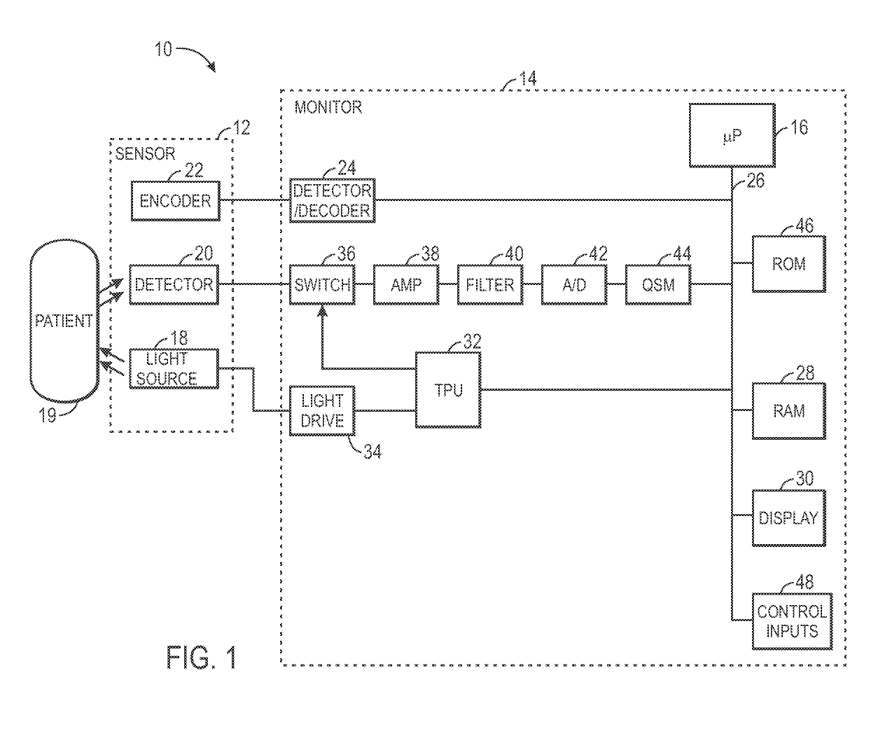

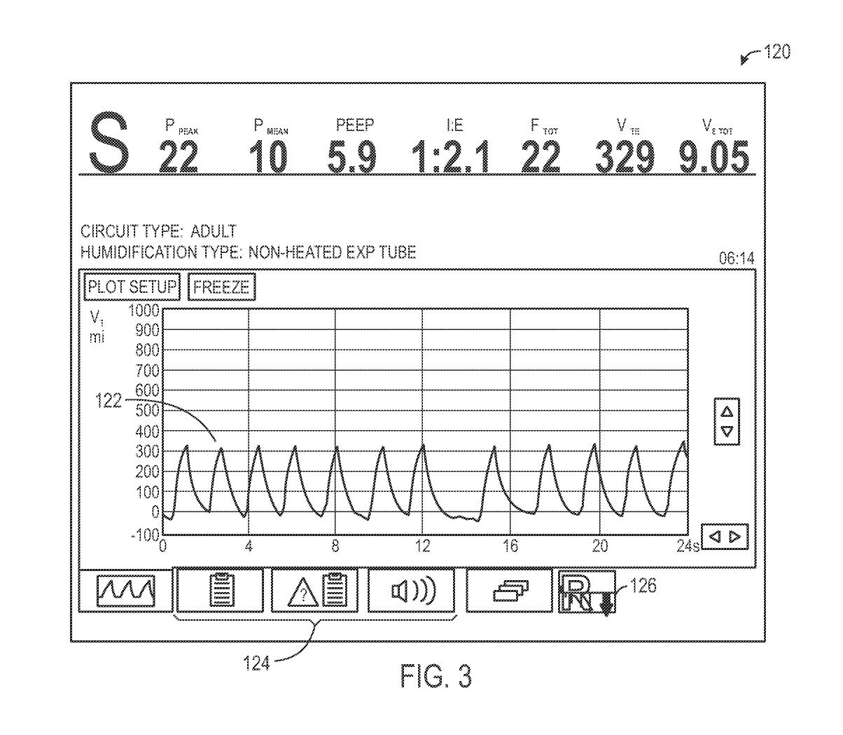

An example of another patient monitoring system 10 is depicted in FIG. 1. The patient monitoring system 10 includes a pulse oximeter 14 with a display 30 and a sensor 12, such as may be available from Covidien LP. The sensor 12 is connected to the pulse oximeter 14. The pulse oximeter 14 includes a microprocessor 16 that may execute data processing algorithms in order to determine a desired physiological parameter to send to the display 30. The physiological parameter of interest may be represented graphically with a sparkline on the display 30 by the microprocessor 16 executing computer instructions stored on a non-transitory machine readable medium. To that end, the following description of the patient monitoring system 10 serves as a basis for describing how the sparkline may be utilized in the display 30 to further the physiological monitoring process with reference to FIGS. 3-10 below. Indeed, the sparkline provides the benefits of displaying a current value and historical physiological parameter values in relation to a baseline and a configurable boundary in a condensed view that is easily ascertainable to a caregiver.

Turning now to the operation of the illustrated patient monitoring system 10, light from a light source 18 passes into a blood perfused tissue of a patient 19 and is scattered and detected by photodetector 20. The sensor 12 containing the light source 18 and the photodetector 20 may also contain an encoder 22 that provides signals indicative of the wavelength of the light source 18 to a detector/decoder 24 to enable the pulse oximeter 14 to select appropriate calibration coefficients for calculating oxygen saturation and/or other physiological parameters. The pulse oximeter 14 includes the microprocessor 16 connected to an internal bus 26. A random access memory (RAM) memory 28 and the display 30 are also connected to the bus 26. Historical physiological parameters may be stored in the RAM 28 and accessed by the microprocessor 16 to display in the sparkline. A time processing unit (TPU) 32 provides timing control signals to light drive circuitry 34, which controls when the light source 18 is illuminated and, if multiple light sources are used, the multiplexed timing for the different light sources. The TPU 32 also controls the gating-in of signals from photodetector 20 through a switching circuit 36. These signals are sampled at the proper time, depending upon which of multiple light sources is illuminated, if multiple light sources are used. The received signal is passed through an amplifier 38, a low pass filter 40, and an analog-to-digital converter 42. The digital data is then stored in a queued serial module (QSM) 44, for later downloading to RAM 28 as QSM 44 approaches its capacity. In one embodiment, there may be multiple parallel paths of separate amplifier, filter and A/D converters for multiple light wavelengths or spectra received.

Based on the value of the received signals corresponding to the light received by detector/decoder 24, the microprocessor 16 will calculate the desired blood parameters, such as blood oxygen saturation, using various algorithms. These algorithms may require coefficients, which may be empirically determined, corresponding to, for example, the wavelengths of light used. These and other parameters, constants, and so forth, may be stored in a read only memory (ROM) 46. In a two-wavelength system, the particular set of coefficients chosen for any pair of wavelength spectra is determined by the value indicated by encoder 22 corresponding to a particular light source in a particular sensor 12. Additionally, a variety of control inputs 48 may be utilized in the calculation of the desired blood parameters. Control inputs 48 may be, for instance, a switch on the pulse oximeter, a keyboard, or a port providing instructions from a remote host computer. Furthermore, any number of methods or algorithms may be used to determine a patient's pulse rate, blood oxygen saturation, or any other desired physiological parameter.

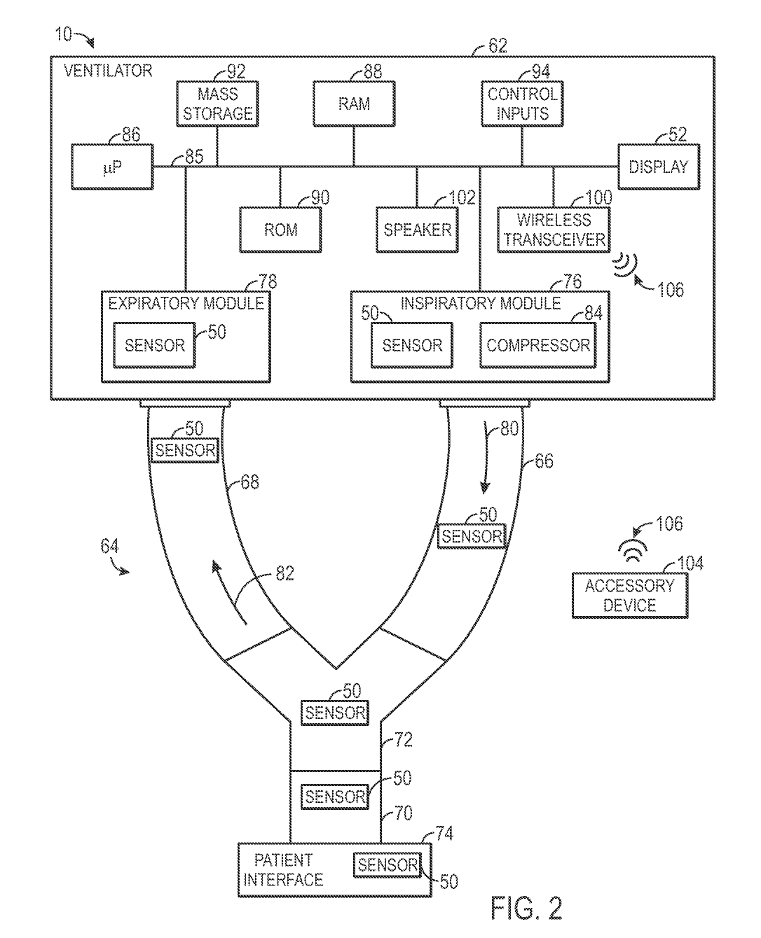

As mentioned above, the envisioned sparkline may be implemented in any number of patient monitoring systems 10, but the focus of the remaining disclosure relates to utilizing a sparkline to display one or more physiological parameters in a ventilator 62. FIG. 2 is a block diagram illustrating an embodiment of such a patient monitoring system 10 including sensors 50 and the ventilator 62 with a display 52, such as may be available from Covidien LP. The ventilator 62 may monitor one or more breathing characteristics of a patient and use the breathing characteristics to set and adjust one or more ventilation parameters. A caregiver may monitor the display 52 on the ventilator 62 to determine the status of the patient and take appropriate action. To that end, the following description of the patient monitoring system 10 serves as a basis for describing how the sparkline may be utilized in the display 52 on the ventilator 62 to further the physiological monitoring process. Indeed, the sparkline may provide several benefits including displaying color-coded values, boundaries, and/or trending historical physiological parameters (e.g., blood oxygen saturation, respiration rate, tidal volume, positive end expiratory pressure, etc.) in a compact and easy to ascertain graphic on a crowded ventilator 62 GUI.

The patient monitoring system 10 may include the ventilator 62 connected to a respiratory circuit 64. The respiratory circuit 64 may be in fluid communication with a source of respiratory gas and may enable one-way flow of inspired gases towards the patient and one-way flow of expired gases away from the patient. In particular, the respiratory circuit 64 may include an inspiratory conduit 66, an expiratory conduit 68, and a patient conduit 70. The inspiratory, expiratory, and patient conduits 66, 68, and 70, may be connected to one another by a Y-connector (i.e., a “wye” connector) 72, which may be connected to a patient interface 74. The patient interface 74 may be any suitable patient interface, such as an endotracheal tube, a tracheostomy tube, or a breathing mask placed over the nose and/or mouth of the patient. Furthermore, the patient monitoring system 10 may include any number of connectors or medical tubing to provide the respiratory gas from the source to the lungs.

The ventilator 62 may include an inspiratory module 76 and an expiratory module 78 for circulating respiratory gases to and from the patient via the respiratory circuit 64 and the patient interface 74. Accordingly, the inspiratory module 76 may be coupled to the inspiratory conduit 66 for providing respiratory gases, represented by arrow 80, and the expiratory module 78 may be coupled to the expiratory conduit 68 for receiving respiratory gases, represented by arrow 82. As used herein, the respiratory gas may be air, oxygen, nitrogen, carbon dioxide, vaporized water, vaporized medicines, or any combination thereof. The inspiratory module 76 may be configured to receive a source of respiratory gas and to pressurize the respiratory gas via a compressor 84. Furthermore, the inspiratory and expiratory modules 76 and 78 may include various suitable components, such as circuitry, valves, filters, tubing, and/or sensors. In one embodiment, the inspiratory and expiratory modules 76 and 78 may be coupled to an internal bus 85 and controlled by a microprocessor 86 to regulate the pressure and/or flow of the respiratory gas delivered and removed.

The microprocessor 86 may access and execute coded instructions, such as for implementing algorithms, from one or more storage components of the ventilator 62, such as a RAM 88, ROM 90, and/or a mass storage device 92. For example, code encoding executable algorithms may be stored in the RAM 88, the ROM 90, and/or the mass storage device 92 (such as a magnetic or solid state hard drive or memory or an optical disk or memory) and accessed and operated according to microprocessor 86 instructions using stored data. In certain embodiments, the RAM 88, the ROM 90, and/or the mass storage device 92 may store information related to historical physiological parameters, one or more settings of the ventilator 62, one or more coefficients or equations for calculating patient physiological parameters, and patient data. For example, the information related to historical physiological parameters may be accessed and displayed by the display 52 in the sparkline, which will be described in detail below. In addition, patient data such as normal values or ranges of respiratory resistance and compliance for various patient populations may be stored. The microprocessor 86 may also receive information related to ventilation settings and/or patient data from a caregiver via one or more control inputs 94. For example, a caregiver may input a patient's gender, age, weight, ideal body weight, and/or condition (e.g., asthma, emphysema, chronic obstructive pulmonary disease, acute respiratory distress syndrome, etc.) which may be used in the selection of the normal values of respiratory resistance and compliance, as well as alarm conditions.

Additionally, the microprocessor 86 may receive information from one or more sensors 50 of the patient monitoring system 10, as will be described in more detail below. In certain embodiments, the microprocessor 86 may also receive information related to patient physiological parameters from other medical devices (e.g., a pulse oximeter, an electrocardiography device, and/or an electroencephalogy (EEG) device) via a wireless transceiver 100. The received information may be stored in the RAM 88, the ROM 90, and/or the mass storage device 92 and may be used in calculations for determining one or more physiological parameters of a patient relating to respiratory function. The ventilator's 62 display 52 and/or speaker 102 may be used to convey information about the calculated physiological parameters and/or ventilation parameters or settings to the caregiver. In particular, the display 52 may be used to display the sparkline to provide the caregiver values, boundaries, and/or historical information for physiological parameters at a glance on a crowded ventilator 62 GUI. Furthermore, the wireless transceiver 100 may be configured to transmit information to one or more accessory devices 104 via wireless communication 106 to enable the caregiver to remotely monitor the calculated physiological parameters and/or the ventilation parameters. For example, the one or more accessory devices 104 may include a remote computer (e.g., located at a nurse's station), a pager, a smart phone, a smart watch, a laptop computer, a handheld computing device, or a cloud computing device.

As noted above, the microprocessor 86 may calculate the one or more physiological parameters based in part upon signals received from the one or more sensors 50 in the patient monitoring system 10. The sensors 50 may obtain signals related to the flow and/or pressure of the supplied and returned respiratory gases, which may be indicative of the patient's respiratory function and in particular, of the patient's effort. Accordingly, any suitable sensor 50 for determining flow, pressure, nerve impulses, concentrations of components in the patient's respiratory gas, or any other desired parameter may be used. For example, the sensors 50 may be pressure sensors, flow sensors, electroencephalogy (EEG) sensors, neural sensors, and/or optical sensors. Additionally, the sensors 50 may generate signals related to certain physiological parameters, such as pressure and flow, which may be used by the processor 86 to derive other physiological parameters. For example, the microprocessor 86 may be configured to derive exhaled tidal volume, inhaled tidal volume, inspiratory time, expiratory time, a ratio of inspiratory time to expiratory time (I:E), respiratory rate, peak inspiratory pressure, positive end-expiratory pressure, plateau pressure, alveolar pressure, inspiratory reserve volume, expiratory reserve volume, vital capacity, functional residual capacity, respiratory resistance, respiratory compliance, and/or any other physiological parameter. In particular, the microprocessor 86 may be configured to integrate the determined inspiratory flow and expiratory flow to derive the inhaled tidal volume and exhaled tidal volume, respectively. Additionally, the microprocessor 86 may be configured to graphically represent one or more physiological parameter in the form of the sparkline on the display 52, which will be described in more detail below.

As mentioned above, ventilator 62 GUIs often include multiple visual elements, such as charts, graphs, or readings, which display physiological parameters. The combined effect of the many visual elements creates a crowded GUI that may make it hard to read certain parameters on the ventilator's 62 display 52. As such, the present techniques disclose, among other things, incorporating a sparkline representing one or more physiological parameters on a patient monitoring system 10, such as the ventilator 62. The sparkline may be a graphic, such as a word-sized graphic, that includes a graph/chart, numbers, percentages, letters, and so forth. The sparkline referred to herein may provide, among other things, regional oximetry information to a caregiver and is small enough to be incorporated into otherwise crowded GUIs. An example of a regional oximeter is the INVOS system from Covidien LP (Boulder, Colo.).

For example, FIG. 3 illustrates an embodiment of a ventilator GUI 120 including a sparkline 126. The ventilator GUI 120 includes many different physiological parameters (e.g., exhaled tidal volume, inhaled tidal volume, inspiratory time, expiratory time, a ratio of inspiratory time to expiratory time (I:E), respiratory rate, peak inspiratory pressure, positive end-expiratory pressure, plateau pressure, alveolar pressure, inspiratory reserve volume, expiratory reserve volume, vital capacity, functional residual capacity, respiratory resistance, respiratory compliance, and/or any other physiological parameter, etc.) represented by various visual elements. For example, a graph 122 and virtual buttons 124 are incorporated in the ventilator GUI 120. The virtual buttons 124 may represent sound, alarms, user settings, and so forth. As can be seen, the sparkline 126 may be incorporated into the crowded ventilator GUI 120 in order to provide additional physiological parameters to a caregiver. The sparkline 126 may be small and may not take up a lot of space on the display. Indeed, the illustrated sparkline 126 may be approximately the same size as the virtual buttons. It is envisioned that the sparkline 126 may be reduced in size as small as possible to still display the physiological parameter(s) in a comprehensible way.

However, the sparkline 126 may be configured to provide a caregiver with a lot of information about a patient at a glance by utilizing trending historical data, boundaries, color-coding, channel indication (e.g., body region being measured), among other features that will be described in more detail below. The sparkline 126 is particularly useful for displaying physiological parameters that are better understood by looking at several readings over a period of time as opposed to just looking at the most recent or current reading. Indeed, some physiological parameters may only make sense by interpreting the changes between readings. For example, when measuring regional oxygen saturation, it is beneficial to see the changes in values from an initial reading over time. Thus, the sparkline 126 may be utilized to display a baseline reading (e.g. initial reading) in conjunction with readings over a period of time in order for a caregiver to interpret any differences.

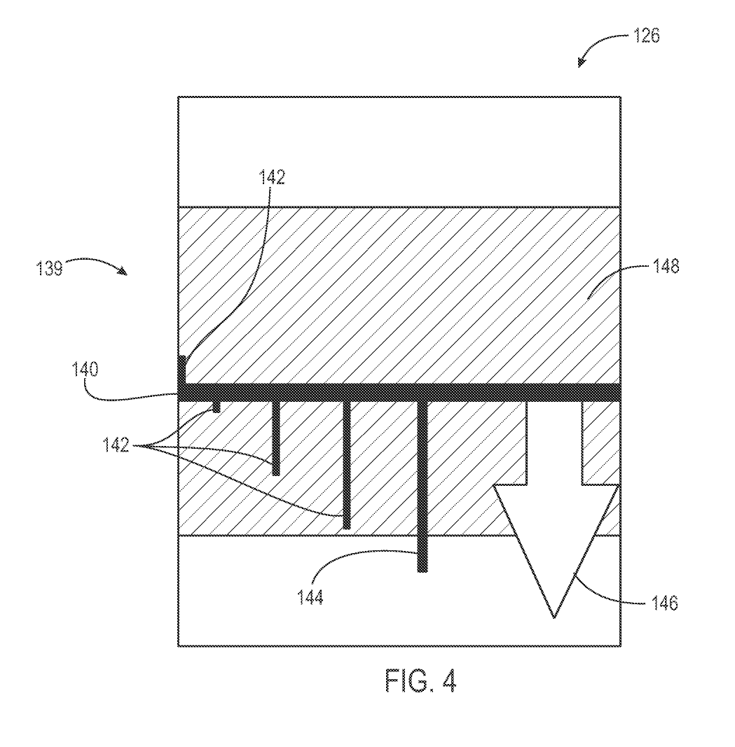

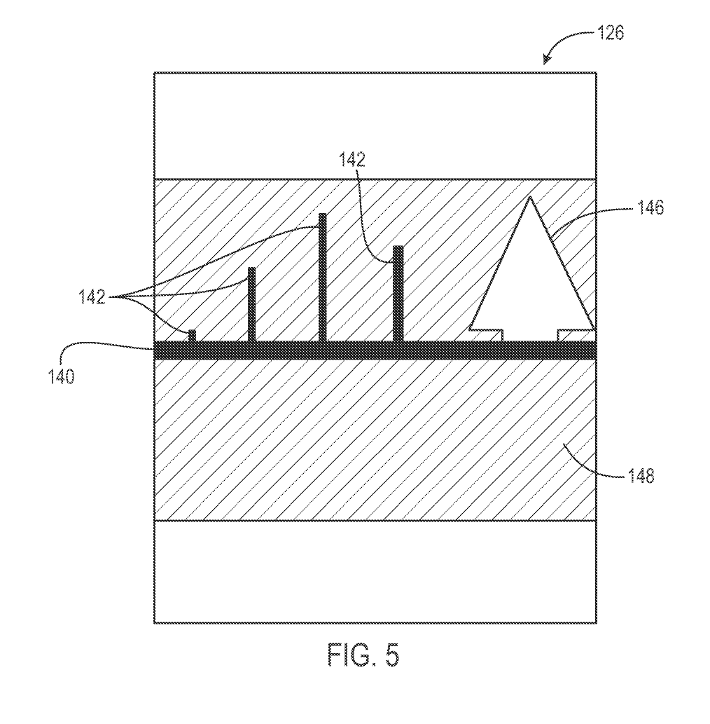

An embodiment of such a sparkline 126 is depicted in FIG. 4. The sparkline 126 displays trending historical physiological parameters with prior values that do and do not exceed a boundary and a current value that exceeds the boundary. As illustrated, one such embodiment of the sparkline 126 may be a bar graph 139 but is not so limited. The bar graph 139 that is illustrated consists of a background area 148, a baseline 140, prior non-exceeding values of a physiological parameter 142, a prior exceeding value of the physiological parameter 144, and a most recent or current value of the physiological parameter 146. The most recent or current value of the physiological parameter 146 is represented by an arrow in the illustrated embodiment, but other embodiments may incorporate any indicator or symbol as to represent the most recent value of the physiological parameter 146. When a new most recent value is measured, the current most recent value of the physiological parameter 146 displayed on the bar graph 139 shifts to the left on the bar graph 139 and becomes a prior value represented by a bar (e.g., vertical line). Accordingly, the prior values of the measured physiological parameter that were already displayed on the bar graph 139 also shift to the left. In alternate embodiments, the most recent value 146 may be on the left side of the bar graph 139 and the prior values (142 and 144) may extend to the right as subsequent readings are taken. Further, the values may be above or below the baseline 140. In some embodiments, values extending above the baseline 140 may represent a positive value change from the baseline 140 value and values extending below the baseline 140 may represent a negative value change from the baseline 140 value. For example, FIG. 4 depicts several prior negative values (142 and 144) extending below the baseline 140, whereas FIG. 5 depicts several prior non-exceeding positive values 142 extending above the baseline 140.

Returning to FIG. 4, the number of readings present on the bar graph 139 at any given time may be configured by the user or the number may be automatically set. For example, the user may configure the readings by selecting a reading interval or the reading interval may be automatic (e.g., default) based on the amount or rate of change of the targeted physiological parameter value between readings. The configuration of the reading interval may involve displaying all readings of the targeted physiological parameter by the patient monitoring system 10 over a length of time, such as the last thirty minutes, hour, or several hours. In certain embodiments, the bars (e.g., vertical lines) may be offset along the baseline 140 (e.g., horizontal line) relative to one another and/or the most recent or current value depending on a length of time that has elapsed between the readings. Alternatively, the number of bars may be set to not display more than a certain number of readings, such as five, ten, fifteen, and so forth. The above examples of how the bar graph 139 may be configured are illustrative only and should not be construed as limiting the present disclosure. It should be noted that other configurations of how many prior readings (e.g., bars in this embodiment) and what length of time the bar graph 139 displays are contemplated in additional embodiments.

The background area 148 may be configured to be any color and represents a boundary, which may also be configured by a user. The boundary may be pre-configured to be 20% of the baseline 140 or any other percentage. Therefore, the background area 148 extends 20% above and 20% below the baseline 140. It should be noted that the user may configure the boundary to be any percentage of the baseline 140 and the background area 148 would adjust based on the configuration. The boundary represents the amount of variation from the baseline 140 that may be acceptable for the value of a certain physiological parameter without raising concerns. For example, in the illustrated embodiment, if oxygen saturation is being measured on an adult, the baseline may be set at 85%. Thus, if the patient's blood oxygen saturation is 21% less than the baseline, which would be 64%, the 20% boundary would be exceeded and there may be cause for concern (e.g. impaired mental function in an average patient).

The background color may be initially set to any color by the user. If the user does not choose an initial color for the background area 148, the default color may be green, indicating that there have been no readings that exceeded the boundary yet. However, the default color may be any suitable color. In certain embodiments, if the boundary is never exceeded by a value from a reading, the background area 148 may remain the default color. For example, if none of the prior or current values of physiological parameters exceeded the 20% boundary, then the background area 148 may be green. However, if one of the prior values did exceed the boundary, then the background area 148 may change colors. In the illustrated embodiment, prior exceeding reading 144 did exceed the boundary and, thus, the background area 148 may be red or any color chosen by a user to represent that the boundary has been exceeded. In some embodiments, when the most recent or current value returns to within the acceptable boundary, the background area 148 may change back to the color indicating that the boundary is not exceeded.

Further, the most recent or current value 146 may also be color-coded. For example, in the illustrated embodiment, the most recent or current value of the measured physiological parameter 146 exceeds the boundary by extending beyond the background area 148. Thus, the most recent or current value 146 would be the color that is set by the user to represent that a value exceeds the boundary. By default, the exceeding color may be set to red. The prior values of the physiological parameter (142 and 144) on the bar graph 139 may also be color-coded based on whether they exceeded the boundary. As will be described below, the result of the foregoing color-coding description means that in certain embodiments the background area 148 may be one color indicating that the boundary has or has not been exceeded in the past, and any prior non-exceeding values 142, prior exceeding values 144, and the most recent or current value 146 may be another color indicating whether they did or did not exceed the boundary.

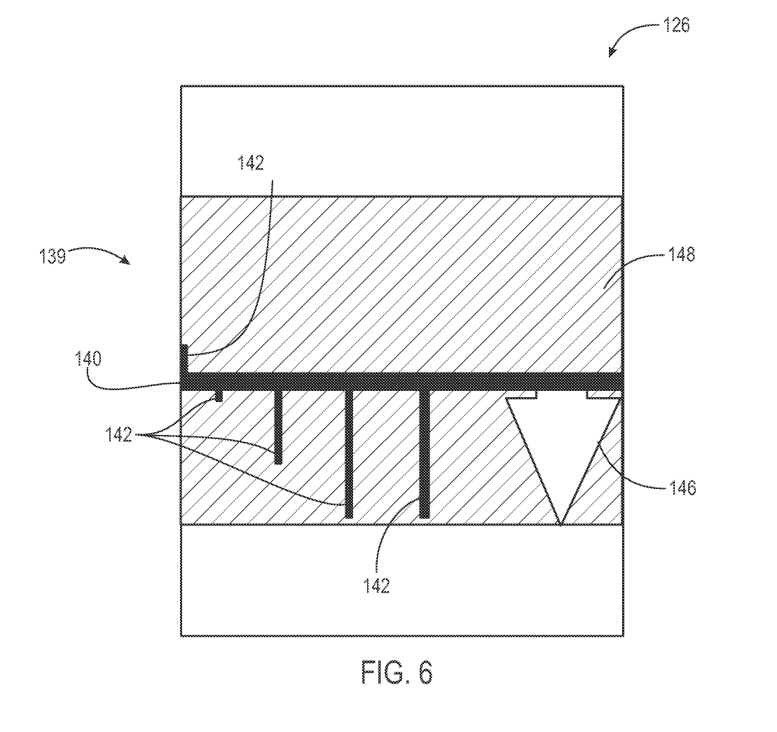

Another embodiment of a sparkline 126 displaying trending historical physiological parameters with prior values that do not exceed a boundary and a current value that does not exceed the boundary is depicted in FIG. 6. The illustrated sparkline 126 includes a bar graph 139 which includes a background area 148, a baseline 140, prior non-exceeding values of a physiological parameter 142, and a most recent or current value of the physiological parameter 146. As previously discussed, the background area 148 represents a boundary that may be configured by a user but may have a default value set to 20% of the baseline 140. In the illustrated embodiment, the prior non-exceeding values 142 did not exceed the boundary and the most recent or current value 146 does not exceed the boundary. Therefore, the background area 148 would be the color indicating that the prior values or most recent value have not exceeded the boundary. For example, in some embodiments, the background area 148 may be green. Additionally, since the most recent or current value 146 does not exceed the boundary, the arrow that represents the most recent or current value 146 would be the color indicating that the most recent or current value 146 does not exceed the boundary (e.g., green or any suitable color). Likewise, the prior non-exceeding values 142 may be color-coded as mentioned above and, in this example, they would be the color indicating that the boundary was not exceeded (e.g., green or any suitable color).

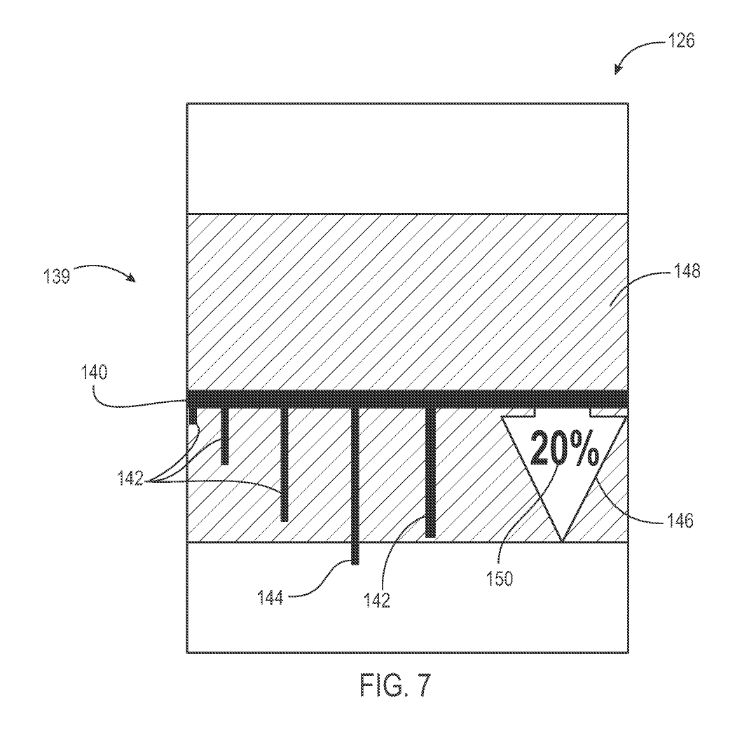

In another embodiment, the sparkline 126 may display trending historical physiological parameters and include a numerical percentage 150, as shown in FIG. 7. The illustrated sparkline 126 includes a bar graph 139 which includes a background area 148, a baseline 140, prior non-exceeding values of a physiological parameter 142, and a most recent or current value of the physiological parameter 146. In this example, there are several non-exceeding values 142 that were within the acceptable boundary (e.g., 20% of the boundary). However, there was one exceeding value 144 that was greater than the boundary. Additionally, the most recent or current value 146 is within the configured boundary as may be seen by the fact that the arrow indicator does not extend beyond the background area 148. Further, the numerical percentage 150 provides a numerical reading to the caregiver of how much the value of the physiological parameter differs from the baseline 140.

As can be seen, the most recent value 146 is 20% below the baseline, as displayed by the numerical percentage 150. This provides the benefit of allowing the caregiver to know the exact percentage of how much the current value of the physiological parameter 146 differs from the baseline 140. As shown in the illustrated sparkline 126, a prior value 144 did exceed the boundary. Therefore, the background area 148 may be the color indicating that the prior values or most recent or current value exceeded the boundary. For example, in some embodiments, the background area may be red. On the other hand, the most recent or current value 146 did not exceed the boundary. Therefore, the arrow indicator may be the color indicating that the boundary was not exceeded (e.g., green or any suitable color). In some embodiments, as mentioned, each bar, or other symbol representing prior values, may be color coded as well. For example, the prior non-exceeding readings 142 may be the color indicating the boundary was not exceeded (e.g., green) and the prior exceeding readings may be the color indicating that the boundary was exceeded (e.g., red).

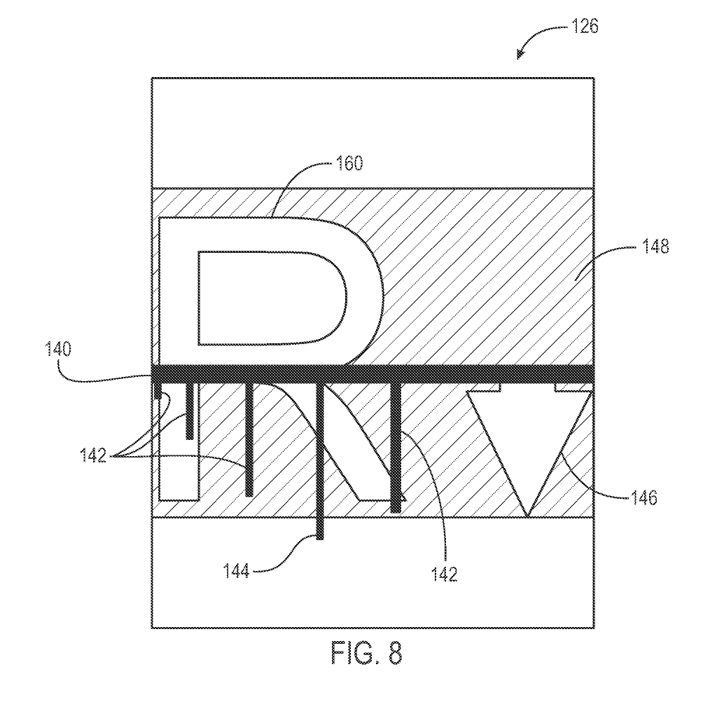

Another embodiment of the sparkline 126 displaying trending historical physiological parameters is displayed in FIG. 8. However, in this embodiment, the sparkline 126 includes a channel indicator 160. In certain embodiments, the physiological parameter may be a regional oxygen saturation level for a particular region of the body. Other examples of physiological parameters that may benefit from the sparkline 126 may include pulse rate, respiration rate, tidal volume, positive end expiratory pressure, and the like. Thus, the channel indicator 160 indicates the channel (e.g., body region) being monitored by the patient monitoring system 10. The channel indicator 160 may be a letter, symbol, or any combination thereof. Further, the channel indicator 160 may be any size or color and may be displayed in the background area 148.

In some embodiments the channel indicator 160 is a large letter (relative to the sparkline's 126 total size) that may be selectable by the user. In particular, the letter “R” may indicate the right cerebral channel, the letter “L” may indicate the left cerebral channel, the letter “K” may indicate the kidney, the letter “G” may indicate the gut, and so forth. It should be appreciated that the channel indicator 160 may be configured by a user. In the illustrated sparkline 126, the channel indicator 160 is set to “R”, indicating that the right cerebral channel is being monitored.

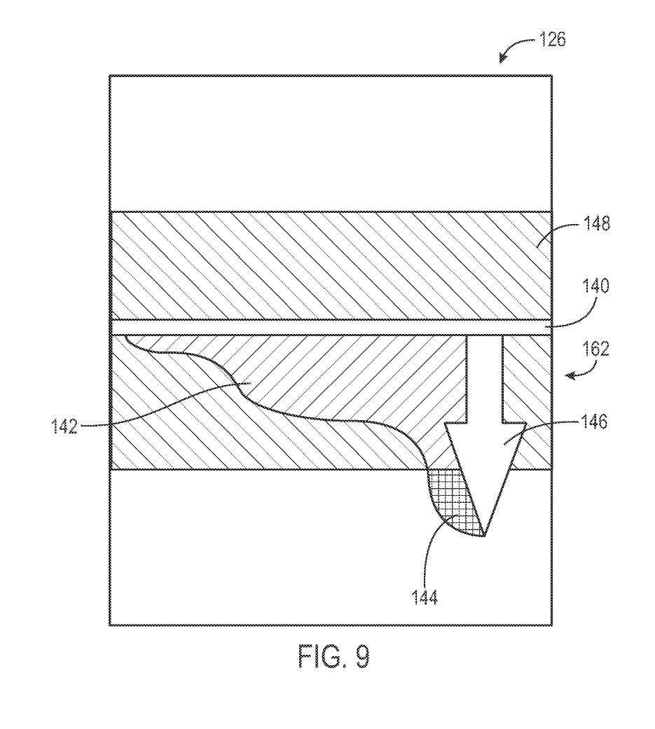

It should be appreciated that the display 30 may display any number of sparklines 126 (e.g., 1, 2, 3, 4, 5) representing any number of physiological parameters in any number of channels (e.g., right cerebral channel, left cerebral channel, kidney, gut). For example, in some embodiments, the display 30 may show two sparklines 126 representing the same or different physiological parameters from two different channels, such as the right cerebral channel and the left cerebral channel. Thus, a first and second value of one or more physiological parameters may be calculated from a first and second channel, and the first and second values may be displayed by a first and second sparkline 126 corresponding to the values from the first and second channels, respectively. As mentioned, the sparkline 126 may be selectable by the user, which may mean that if the user clicks on the sparkline 126, the sparkline 126 may display more detailed information and/or additional information, as will be discussed in more detail below with reference to FIG. 9.

In yet another embodiment, FIG. 9 illustrates a sparkline 126 displaying trending historical physiological parameters including a waveform representing prior values (142 and 144). The illustrated sparkline 126 includes a graph 162, which includes a background area 148, a baseline 140, prior non-exceeding values of a physiological parameter 142, prior exceeding values 144, and a most recent or current value of the physiological parameter 146. However, as may be seen, the prior values are depicted in waveform, as opposed to bar form. The portion of the wave that represents the prior non-exceeding values 142 may be shaded the color indicating that the boundary was not exceeded, and the portion of the wave that represents the prior exceeding values 144 may be shaded the color indicating that the boundary was exceeded. As may be appreciated, the prior values may be represented in any suitable form capable of distinguishing exceeding and non-exceeding readings through the use of color-coding or other means. It should be noted that, regardless of whether color is utilized in the sparkline 126, use of the background area 148 will enable a caregiver to determine whether a prior value or the current value has exceeded the boundary because the values will extend beyond the background area 148. Thus, the techniques disclosed herein are enabled on a patient monitoring system's display that is incapable of displaying color.

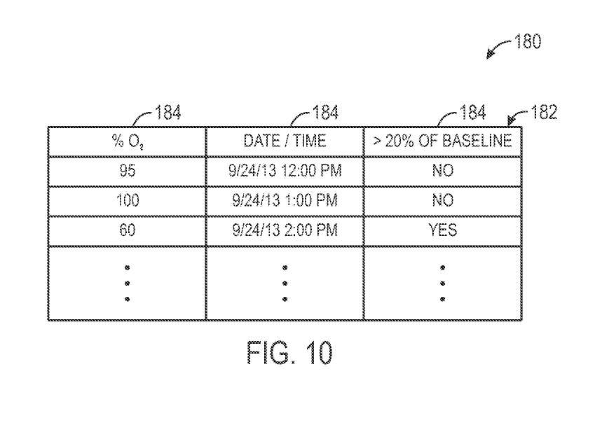

As mentioned above and shown in FIG. 10, in certain embodiments, more detailed and/or additional information may be displayed on a screen 180 when the sparkline 126 is clicked. A user may select the sparkline 126 by clicking on any portion of it (e.g., the channel indicator 160, the bar graph 139, the graph 126, the prior non-exceeding values of a physiological parameter 142, the prior exceeding values 144, the background area 148, and so forth). In some embodiments, when the user selects the sparkline 126 by clicking on it, a more detailed drill-down screen 180 may be displayed that contains a chart 182. For example, the chart 182 may contain columns 184 that represent the type of measurement (e.g., regional oxygen saturation) being analyzed on the patient monitoring system 10, the date and time the reading was taken, whether the value is greater than the baseline 140, and so forth. It should be appreciated, that the drill-down screen 180 may be configurable by a user and the chart's 182 columns 184 may be modified to contain any relevant information. Further, the drill-down screen 180 may contain any number of visual elements including, but not limited to, graphs, charts, tables, and so forth.

While the embodiments set forth in the present disclosure may be susceptible to various modifications and alternative forms, specific embodiments have been shown by way of example in the drawings and have been described in detail herein. However, it should be understood that the disclosure is not intended to be limited to the particular forms disclosed. The disclosure is to cover all modifications, equivalents, and alternatives falling within the spirit and scope of the disclosure as defined by the following appended claims.

What is claimed:

1. A system comprising:
a display; and
a processor configured to calculate and to cause the display of one or more physiological parameters of a patient on the display, the processor being configured to cause the display of at least one physiological parameter of the one or more physiological parameters as a sparkline on the display;
wherein the sparkline comprises a horizontal line representative of a baseline value of the at least one physiological parameter, one or more vertical lines representative of one or more past values of the at least one physiological parameter that extend perpendicularly from the horizontal line, and a vertical arrow representative of a current value of the at least one physiological parameter.

2. The system of claim 1, wherein the one or more vertical lines are representative of a past percent change in the value of the at least one physiological parameter relative to the baseline value.

3. The system of claim 2, wherein the vertical arrow is representative of a current percent change in the value of the at least one physiological parameter relative to the baseline.

4. The system of claim 1, wherein the horizontal line comprises a first side and a second side, wherein if any vertical line or the vertical arrow extends from the first side it represents a positive value change for the at least one physiological parameter, and if any vertical line or the vertical arrow extends from the second side it represents a negative value change for the at least one physiological parameter.

5. The system of claim 1, wherein the one or more past values of the at least one physiological parameter are within a period of time preceding the current value.

6. The system of claim 5, wherein the one or more vertical lines are axially offset along the horizontal line relative to one another, and a distance of an offset between adjacent vertical lines or between the vertical arrow and a vertical line represents a time interval between the respective values for the at least one physiological parameter.

7. The system of claim 1, wherein the one or more vertical lines are color-coded to indicate whether any of the past values of the at least one physiological parameter exceeds a boundary value for the at least one physiological parameter or whether any of the past values of the at least one physiological parameter falls within the boundary value and the baseline value.

8. The system of claim 7, wherein the boundary is set to twenty percent of the baseline reading by default.

9. The system of claim 1, wherein the vertical arrow is color-coded to indicate whether the current value of the at least one physiological parameter exceeds a boundary value for the at least one physiological parameter or whether the current value of the at least one physiological parameter falls within the boundary value and the baseline value.

10. The system of claim 1, wherein the sparkline comprises a background having a first color, and the processor is configured to change the first color to a second color to indicate that the current value of the at least one physiological parameter exceeds a boundary value for the at least one physiological parameter and to change the second color back to the first color if the current value of the at least one physiological parameter returns to within the boundary value and the baseline value.

11. The system of claim 10, wherein the at least one physiological parameter is regional oxygen saturation, and the background comprises an indicator of a channel from which a value of the regional oxygen saturation is obtained.

12. The system of claim 1, wherein a respective distance that the one or more vertical lines and the vertical arrow extend perpendicularly from the horizontal line represents a respective amount of change from the baseline value.

13. The system of claim 1, wherein the display comprises a graphical user interface, and the sparkline is configured to be selected by a user to cause display of additional information related to the at least one physiological parameter on the display.

14. The system of claim 1, wherein the system comprises a ventilator, and wherein the processor is configured to display the sparkline on the display along with a plurality of other ventilation parameters.

15. The system of claim 1, wherein the at least one physiological parameter comprises regional oxygen saturation.

16. A method comprising:
calculating a value for at least one physiological parameter of a patient using a processor; and
displaying on a display a sparkline for the at least one physiological parameter, wherein the sparkline comprises a horizontal line representative of a baseline value of the at least one physiological parameter, one or more vertical lines representative of one or more past values of the at least one physiological parameter wherein the one or more vertical lines extend perpendicularly from the horizontal line, and a vertical arrow representative of a current value of the at least one physiological parameter.

17. The method of claim 16, wherein the one or more vertical lines are representative of a past percent changes in the value of the at least one physiological parameter relative to the baseline value, and the vertical arrow is representative of a current percent change in the value of the at least one physiological parameter relative to the baseline.

18. The method of claim 16, wherein the sparkline comprises a background having a first color, and the method comprises changing the first color of the background to a second color to indicate that the current value of the at least one physiological parameter exceeds a boundary value for the at least one physiological parameter and changing the second color of the background back to the first color if the current value of the at least one physiological parameter falls within the boundary value and the baseline value.

19. The method of claim 16, wherein the display comprises a graphical user interface, and the method comprises receiving a selection of the sparkline and displaying additional information related to the at least one physiological parameter on the display in response to the selection.

20. The method of claim 16, comprising calculating a new current value of the at least one physiological parameter, converting the vertical arrow representative of the current value to a new vertical line representative of a new past value of the at least one physiological parameter, and axially shifting the new vertical line along the horizontal line, and displaying a new vertical arrow representative of the new current value adjacent the new vertical line.

21. The method of claim 16, changing a color of the vertical arrow from a first color to a second color to indicate that the current value of the at least one physiological parameter exceeds a boundary value for the at least one physiological parameter, and changing the second color of the vertical arrow back to the first color if the current value of the at least one physiological parameter falls within the boundary value and the baseline value.

22. The method of claim 16, wherein calculating the value for at least one physiological parameter comprises calculating first and second values of one or more physiological parameters from first and second channels, and wherein displaying the sparkline comprises displaying first and second sparklines corresponding to the values from the first and second channels, respectively.

23. A non-transitory machine readable computer medium comprising computer instructions configured to:
display a sparkline on a patient monitoring system's graphical user interface, wherein the sparkline comprises:
a graphic comprising:
a horizontal line representing a baseline reading of a physiological parameter,
a color-coded background area surrounding the horizontal line and creating a boundary that is a configurable percentage of the baseline reading, and
a color-coded first shape extending perpendicularly from the horizontal line, the first shape representing a current reading of the physiological parameter,
wherein the color-coded background area changes color based on whether the color-coded first shape exceeds the configurable boundary, and
wherein the color-coded first shape changes color based on whether it exceeds the configurable boundary; and
update the sparkline when a subsequent reading is taken, wherein updating the sparkline comprises displaying a color-coded second shape representing the previous current reading, changing the color-coded first shape to be representative of the subsequent reading, and displaying the color-coded second shape adjacent to the changed color-coded first shape.
